(19) United States
(12) Patent Application Publication
(10) Pub. No.: US 2008/0021324 A1
(43) Pub. Date: Jan. 24, 2008

(54) Ultrasonic examination apparatus

(75) Inventor: Yasuhiro Seto, Ashigarakami-gun (JP)

Correspondence Address: SUGHRUE MION, PLLC, 2100 PENNSYLVANIA AVENUE, N.W., SUITE 800, WASHINGTON, DC, 20037, US

(73) Assignee: FUJIFILM Coporation, Minato-ku (JP)

(21) Appl. No.: 11/826,241

(22) Filed: Jul. 13, 2007

(30) Foreign Application Priority Data
Jul. 18, 2006 (JP) 2006-195377

(51) Int. Cl. A61B 8/00

(52) U.S. Cl. 600/447

(57) ABSTRACT

An ultrasonic examination apparatus in which a number of wirings for connecting a probe to an ultrasonic examination apparatus main body can be reduced, and sidelobes can be suppressed even when an ultrasonic beam is deflected. The ultrasonic examination apparatus includes: a probe having a multirow array formed by arranging plural element rows in parallel with one another, each element row including one-dimensionally arranged ultrasonic transducers, and switches for opening and closing electric connections between respective adjacent two elements in each element column of the multirow array to form element groups; a system control unit for controlling the switches according to a transmission/reception direction of an ultrasonic beam; a drive signal generating unit for generating drive signals to be respectively supplied to the element groups; and signal processing units for processing reception signals respectively outputted from the element groups to generate an image signal.

### BACKGROUND OF THE INVENTION

1. Field of the invention

The present invention relates to an ultrasonic examination apparatus for transmitting ultrasonic waves toward an object to be inspected and receiving ultrasonic echoes from the object to generate ultrasonic images, and specifically, to an ultrasonic examination apparatus including an ultrasonic endoscope in which an ultrasonic transducer array is combined with an endoscope to be inserted into a body cavity of the object for optical observation of the condition within the object.

2. Description of a Related Art

Ultrasonic imaging technologies for generating images showing tissue conditions within an object to be inspected by receiving ultrasonic echoes, that is, ultrasonic waves transmitted toward the interior of the object and reflected by structures (organs and so on) within the object and performing signal processing thereon are widely used in various fields including medical fields. An apparatus for performing ultrasonic examinations (called an ultrasonic examination apparatus, ultrasonic diagnostic apparatus, or the like) is provided with a probe for transmission and reception of ultrasonic waves, and the probe is used in contact with an object to be inspected at the time of imaging. Further, also an ultrasonic endoscope in combination of a probe (an ultrasonic transducer array) for transmission and reception of ultrasonic waves and an endoscope for optical observation of the condition within the body cavity of the object is widely used, and the ultrasonic endoscope is used by being inserted into the object.

In the ultrasonic probe and the ultrasonic endoscope (hereinafter, they are also collectively and simply referred to as “probe”), vibrators (hereinafter, also referred to as “elements”) each having a piezoelectric material with electrodes formed on both sides thereof are generally used as ultrasonic transducers for transmission and reception of ultrasonic waves. When an electric field is applied to the electrodes of the vibrator, the piezoelectric material expands and contracts because of piezoelectric effect and generates ultrasonic waves. Accordingly, an ultrasonic beam focused at a desired depth can be formed by driving plural vibrators while shifting the timing. Further, those vibrators receive propagating ultrasonic waves, expand and contract, and generate electric signals, respectively. These electric signals are used as reception signals of ultrasonic waves.

In such a probe, an arrayed transducer (an ultrasonic transducer array) in which plural elements are arranged used. For example, an array in which plural elements are arranged linearly or circularly in one row in the scan direction (azimuth direction) is called a one-dimensional (1D) array. The transmission position and direction of an ultrasonic beam can be changed by controlling the amplitudes and amounts of delay of drive signals to be applied to the respective elements of the 1D array without change in the position and orientation of the probe itself. Such a scan system is called a phased array system or electronic scan system.

Recently, researches on a phased array (2D array) in which many vibrators are two-dimensionally arranged have been increasingly made. The transmission direction and focal point of an ultrasonic beam can be arbitrarily controlled by transmitting plural ultrasonic waves from a two-dimensional region, and three-dimensional ultrasonic image information (volume data) can be acquired. Thereby, the position, spread, size, and so on of a lesion part can be correctly grasped, and the accuracy of ultrasonic examination can be dramatically improved.

However, since microelements are used in the 2D array, the manufacturing process thereof is microscopic and complicated. Further, the number of wirings increases with increase in the number of elements, and therefore, a problem that a cable connecting the probe and the ultrasonic examination apparatus main body becomes thicker arises. Especially, the thicker cable is a fatal flaw because severe constraints in size are imposed on the ultrasonic endoscope to be inserted into a living body.

As a measure to solve the problem, a so-called multirow array, in which plural 1D arrays are arranged in parallel, attracts attention. Although the number of 1D arrays arranged in the multirow array is not as many as that in a matrix arrangement, an ultrasonic beam focused in two directions can be formed by using vibrators arranged in the two-dimensional region. As a related technology, in Wildes et al., “Elevation Performance of 1.25D and 1.5D Transducer arrays”, (IEEE TRANSACTIONS ON ULTRASONICS, FERROELECTRICS, AND FREQUENCY CONTROL, VOL. 44, NO. 5, SEPTEMBER 1997, pp. 1027-1037), the performance of multirow array, in which the vibrator arrangement in the elevation direction and the wiring method are changed, is studied.

Here, a structure of a general multirow array will be explained with reference to FIG. 15. FIG. 15(a) is a side view showing a multirow array, and FIG. 15(b) is a plan view thereof. The multirow array contains plural elements 902 arranged in 11 rows (row E1 to row E11) on a backing material 901. Further, the respective rows contain 128 elements 902, for example. In the multirow array, the element arrangement direction (scan direction) in the respective element rows is called an azimuth direction, and the direction perpendicular to the azimuth direction is called an elevation direction. Furthermore, the elements 902 are respectively connected to wirings 903.

In such a multirow array, in order to improve the quality of ultrasonic beam by reducing grating lobes, the array is typically designed such that the arrangement pitch of elements in the azimuth direction is equal to or less than the wavelength of transmission ultrasonic waves. On the other hand, with respect to the elevation direction, the elements are typically arranged at an arrangement pitch equal to or more than the wavelength. With the features, a significant advantage that the number of elements and wirings can be drastically reduced is obtained in the multirow array, although the ultrasonic beam quality such as resolving power and the scanning volume remain inferior to those of the matrix arrangement array. That is, downsizing and reduction in costs of the ultrasonic probe and ultrasonic endoscope can be realized. About 10 rows of elements are necessary for obtaining good quality ultrasonic images by using the multirow array.

As a related technology, Japanese Patent Application Publication JP-P2000-139926A discloses an ultrasonic probe including ultrasonic transmitting and receiving means, provided at a leading end of an insertion part to be inserted into a body cavity, for transmitting and receiving ultrasonic beams, a treatment tool lead-out opening from which a treatment tool such as a puncture needle can be led out toward a scan range of ultrasonic beam by the ultrasonic transmitting and receiving means, and ultrasonic deflecting means for deflecting the scan range of ultrasonic beam by the ultrasonic transmitting and receiving means. That is, according to JP-P2000-139926A, ultrasonic vibrators are arranged in three rows and ultrasonic waves with different phases are transmitted from the respective rows for deflection of the scan range of ultrasonic waves, and thereby, the ultrasonic beam is applied to the punctuation needle even when the punctuation needle is bent.

Further, Japanese Patent Application Publication JP-P2004-57460A discloses an ultrasonic diagnostic apparatus having a continuous wave Doppler mode, and the ultrasonic diagnostic apparatus includes a vibrator array having plural vibrating elements arranged in an electronic scan direction and an elevation direction perpendicular to the electronic scan direction, and a transmission and reception control unit for controlling the operation of the plural vibrating elements. In the continuous wave Doppler mode, at least one group of transmission vibrating elements arranged in the electronic scan direction and at least one group of reception vibrating elements arranged in the electronic scan direction are set in different positions from each other in the elevation direction on the vibrator array. That is, according to JP-P2004-57460A, the transmission aperture and the reception aperture are taken wider by alternate arrangement of transmission vibrating element row and reception vibrating element row.

Furthermore, Japanese Patent Application Publication JP-P2003-130859A discloses a phased array driving apparatus for controlling drive of a phased array probe having first to n-th ultrasonic vibrators (“n” is an integral number equal to or more than “2”), and the phased array driving apparatus includes a drive circuit for outputting drive signals for driving the first to n-th ultrasonic vibrators, and timing adjustment means for shifting the timing of the drive signals and providing them as the first to n-th drive signals to the first to n-th ultrasonic vibrators, respectively. That is, according to JP-P2003-130859A, in order to reduce the number of drive circuits, the plural ultrasonic vibrators are driven with shifted timing. Further, the first to n-th ultrasonic vibrators are divided into first to m-th groups (“m” is a natural number less than “n”), and drive signals are selectively provided to the ultrasonic vibrators that belong to those first to m-th groups. That is, the plural ultrasonic vibrators are grouped and the drive timing of the ultrasonic vibrators is controlled with respect to each group, and thus, the total time for controlling drive of all ultrasonic vibrators is shortened.

Meanwhile, since the multirow array has the number of elements in the elevation direction between those of the 1D array and the 2D array, they are also called 1.25D array, 1.5D array, and 1.75D array. According to the definition of Wildes et al., the dimensions of arrays can be explained as in the following (1) to (5).

(1) 1D array: Plural elements are arranged in one row (in the azimuth direction). Accordingly, the aperture diameter in the elevation direction (the element width in this case) is fixed and the focal point of ultrasonic beam is formed by an acoustic lens or the like, and therefore, the focal length is fixed.

(2) 1.25D array: Plural 1D arrays are arranged in parallel. Although the aperture diameter in the elevation direction is variable (one to 11 rows), the focal point of ultrasonic beam is formed by an acoustic lens or the like, and therefore, the focal length is fixed.

(3) 1.5D array: An array in which two elements 902 symmetric with respect to the central axis in the longitudinal direction of the array (e.g., E1-row and E11-row, E2-row and E10-row, . . . ) are connected in parallel (commonly connected to the same wiring), and those elements 902 are driven with the same timing. Accordingly, the aperture diameter in the elevation direction is variable (the one to 11 rows), also the focal length can be dynamically changed by adjusting the drive timing of the elements with respect to each wiring. However, the ultrasonic beam is not deflectable in the elevation direction.

(4) 1.75D array: The constraint that the symmetry with respect to the central axis in the longitudinal direction of the array is removed from the 1.5D array by independently interconnecting the respective elements 902. Thereby, the ultrasonic beam can be deflected in the elevation direction in addition to changing the aperture diameter and the focal length. However, in the elevation direction, the width of the element is larger than the wavelength of the ultrasonic waves, and thus, constraints are imposed on the range where the ultrasonic beam can be deflected, and there is no degree of freedom equal to that in the azimuth direction.

(5) 2D array: The number of elements and the arrangement pitch in the elevation direction are made substantially equal to those in the azimuth direction. Therefore, apodization, the formation of focal point in the three-dimensional space, and the deflection of ultrasonic beam can be perfectly controlled.

Such a multirow array is designed to improve the quality of ultrasonic beams with the less number of element rows. For example, FIG. 16 shows a multirow array having elements in weighted arrangement in the elevation direction. In the multirow array, elements 912-914 arranged on a backing material 911 have widths that are narrower from the central part toward the outer side. Further, wirings 915 are connected to the respective elements 912-914. As the weighted arrangement, methods called Fresnel arrangement, MIAE (Minimum Integrated Absolute time-delay Error) arrangement, and so on are known. Refer to Wildes et al. for details of the Fresnel arrangement and MIAE arrangement.

In the multirow array shown in FIG. 15, in the case where the 1.75D array system is adopted, there is an advantage that the ultrasonic beam can be deflected in the elevation direction. However, in this case, the number of wirings 903 for supplying the drive signals to the elements 902 is necessary as many as the number of elements. For example, when 11 rows of 128 channels of element rows are arranged, the number of signal lines reaches 1408. The larger number of wirings is a fatal flaw in the ultrasonic endoscope to be inserted into the object in view of operability, pain of patients, and so on.

In contrast, as disclosed in JP-P2003-130859A, in the case where the plural elements are grouped and driven, the number of drive circuits and the number of wirings connecting the drive circuits and the plural elements can be drastically reduced. However, according to JP-P2003-130859A, there is a problem that the number of switches for switching the groups increases. For example, when eleven elements are divided into five groups in the elevation direction, five switching switches are provided for the eleven elements, and 55 switches are necessary for one channel. Accordingly, when the respective element rows have 128 channels, the total number of switches reaches 7040. If the large number of switches are provided at the probe side, the probe itself is upsized, and that is a fatal flaw in the ultrasonic endoscope.

On the other hand, when the weighted arrangement shown in FIG. 16 is adopted, good quality ultrasonic beams can be formed with the less numbers of elements and wirings (128 channels×five rows=640). However, if the 1.75D array is adopted in the weighted arrangement for deflection of the ultrasonic beams, a problem in increase of sidelobes arises. This is because the element width and arrangement are optimized in the typical weighted arrangement such that the beam quality becomes the best when the ultrasonic beam is transmitted in the central direction.

### SUMMARY OF THE INVENTION

Accordingly, in view of the above-mentioned points, a purpose of the present invention is to provide an ultrasonic examination apparatus in which a number of wirings for connecting a probe to an ultrasonic examination apparatus main body can be reduced, and the expansion of sidelobes can be suppressed even when an ultrasonic beam is deflected in the elevation direction.

In order to achieve the above-mentioned purpose, an ultrasonic examination apparatus according to one aspect of the present invention includes: a probe having a multirow array formed by arranging plural element rows in parallel with one another, each element row including one-dimensionally arranged ultrasonic transducers, and plural switches for opening and closing electric connections between respective adjacent two elements in each element column of the multirow array to form plural element groups; control means for controlling the opening and closing of the plural switches according to a transmission/reception direction of an ultrasonic beam; drive signal generating means for generating plural drive signals to be respectively supplied to the plural element groups; and signal processing means for processing plural reception signals respectively outputted from the plural element groups to generate an image signal.

According to the present invention, plural elements are grouped in each element column of the multirow array and drive signals are supplied with respect to each group, and therefore, the number of wirings to be connected to the multirow array can be reduced. Thereby, the number of cables connecting the probe to the ultrasonic examination apparatus main body can be reduced, and thus, the operability of the probe can be improved and the physical burden on the patient to be examined can be reduced. Further, the combinations of elements to be grouped can be optimized according to the transmission direction of an ultrasonic beam, and therefore, a good quality ultrasonic beam can be transmitted regardless of the transmission direction of the ultrasonic beam. Thereby, image quality of ultrasonic images can be improved.

### BRIEF DESCRIPTION OF THE DRAWINGS

FIG. 1 is a block diagram showing a configuration of an ultrasonic examination apparatus according to one embodiment of the present invention;

FIG. 2 shows an ultrasonic transducer array to be used in a probe shown in FIG. 1;

FIG. 3 is a block diagram showing a configuration of an ultrasonic examination apparatus main body shown in FIG. 1;

FIGS. 4A and 4B show connecting condition at the time of ultrasonic beam transmission by using the probe shown in FIG. 1;

FIGS. 5A and 5B show states in which ultrasonic beams are transmitted from the ultrasonic transducer array with plural elements in uniform arrangement;

FIG. 6 shows profiles of ultrasonic beams transmitted from the ultrasonic transducer array shown in FIGS. 5A and 5B;

FIGS. 7A and 7B show states in which ultrasonic beams are transmitted from the ultrasonic transducer array with plural elements in weighted arrangement;

FIG. 8 shows profiles of ultrasonic beams transmitted from the ultrasonic transducer array shown in FIGS. 7A and 7B;

FIG. 9 shows profiles of ultrasonic beams transmitted from the ultrasonic transducer array shown in FIGS. 4A and 4B;

FIG. 10 shows an equivalent circuit of a transmission system circuit in the ultrasonic examination apparatus according to the one embodiment of the present invention;

FIG. 11 shows an equivalent circuit of a reception system circuit in the ultrasonic examination apparatus according to the one embodiment of the present invention;

FIG. 12 is a schematic diagram showing an ultrasonic endoscope to which the probe shown in FIG. 1 is applied;

FIG. 13 is a schematic diagram showing a medical image generating apparatus connected to the ultrasonic endoscope shown in FIG. 12;

FIG. 14 is an enlarged schematic diagram showing the leading end of an insertion part shown in FIG. 12;

FIG. 15 shows a general multirow array; and

FIG. 16 shows a multirow array with plural kinds of elements in weighted arrangement.

### DESCRIPTION OF THE PREFERRED EMBODIMENTS

Hereinafter, preferred embodiments of the present invention will be explained in detail with reference to the drawings. The same reference numerals will be assigned to the same component elements and the description thereof will be omitted.

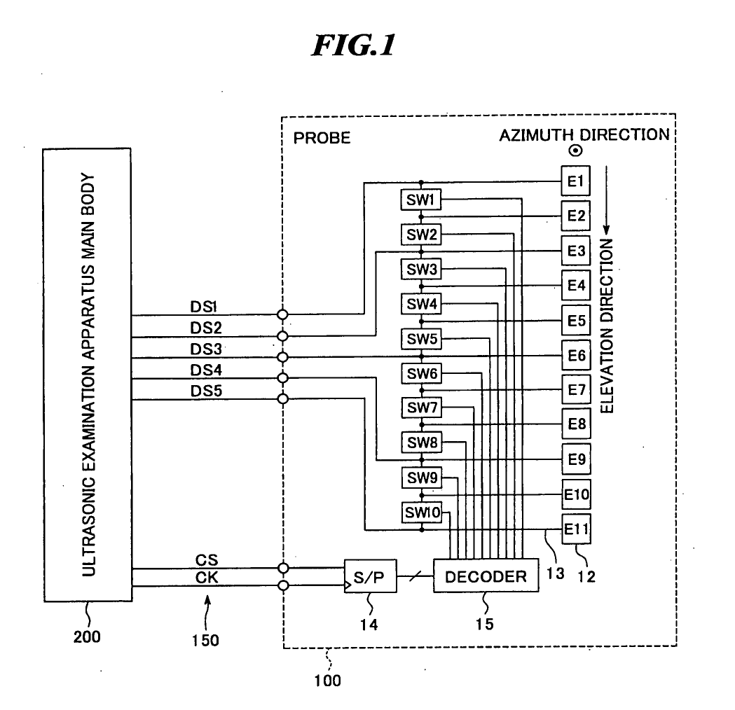

FIG. 1 is a block diagram showing a configuration of an ultrasonic examination apparatus according to one embodiment of the present invention. The ultrasonic examination apparatus includes a probe 100, an ultrasonic examination apparatus main body 200, and a cable 150 for connecting them to each other. The probe 100 is suitable for use in an ultrasonic endoscope examination by being inserted into a body cavity of an object to be inspected so as to observe the condition within the object, but also suitable for typical ultrasonic examination by being in contact with the body surface of the object.

As shown in FIG. 1, the probe 100 includes an ultrasonic transducer array 10 including plural element rows of E1 to E11, plural switches SW1 to SW10, a serial/parallel converter circuit (S/P) 14, and a decoder 15.

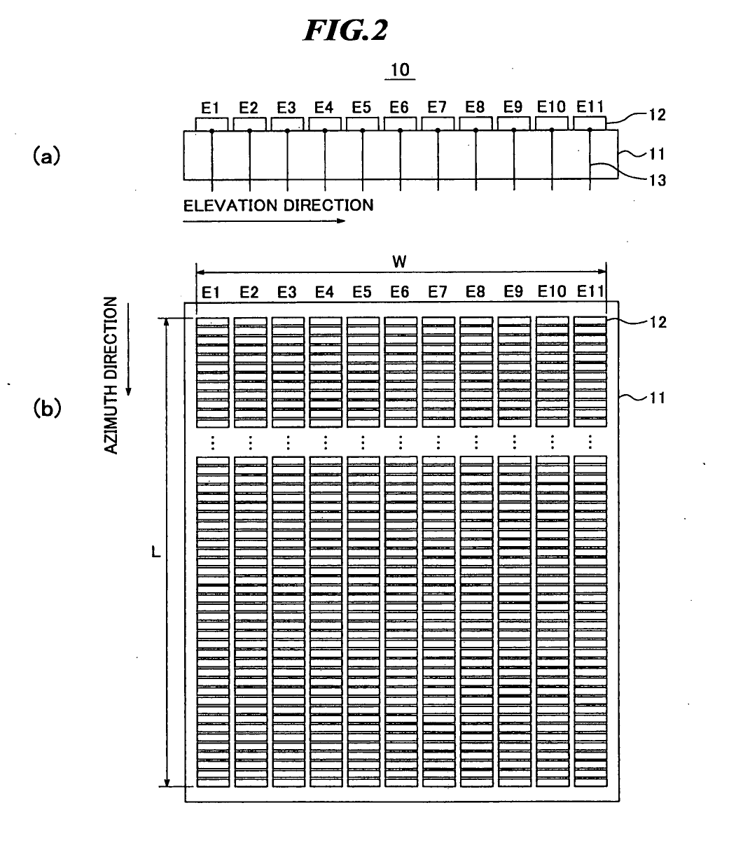

FIG. 2(a) is a side view showing the ultra sonic transducer array 10 shown in FIG. 1, and FIG. 2(b) is a plan view thereof. As shown in FIG. 2(b), the respective element rows E1 to E11 contain 128 ultrasonic transducers (hereinafter, also simply referred to as “elements”) 12 arranged at a predetermined pitch on a backing material 11. Further, as shown in FIG. 2(a), each element 12 is connected to a wiring 13. As below, the arrangement direction of elements in each element row (scan direction) is referred to as “azimuth direction”, and the direction perpendicular thereto (the arrangement direction of elements in each element column) is referred to as “elevation direction”.

The backing material 11 is formed of a material having large acoustic attenuation such as an epoxy resin containing ferrite powder, metal powder, or PZT powder, or rubber containing ferrite powder, and disposed for supporting the elements 12 and promoting attenuation of unwanted ultrasonic waves generated by the ultrasonic transducer array 10.

Each element 12 is a vibrator having a piezoelectric material such as PZT (lead (Pb) zirconate titanate) with electrodes formed on both sides thereof, and expands and contracts to generate ultrasonic waves when a voltage is applied thereto. Further, each element 12 expands and contracts because of ultrasonic waves propagating from the object and generates a voltage. This voltage is outputted to the ultrasonic examination apparatus main body 200 as a reception signal. One electrode of each element 12 is supplied with corresponding one of drive signals DS1 to DS5, and the other electrode is supplied with a common potential (the ground potential in the embodiment).

An acoustic matching layer may be further provided as an upper layer of the ultrasonic transducer array 10 for efficiently propagating the ultrasonic waves transmitted from the ultrasonic transducers within the object by resolving the mismatch of acoustic impedances between the object as a living body and the ultrasonic transducers. The acoustic matching layer is formed of Pyrex (registered trademark) glass or an epoxy resin containing metal powder, which easily propagates ultrasonic waves, for example.

Typically, the arrangement pitch of the elements 12 in the azimuth direction is designed so as to be equal to or less than the half of the wavelength of transmission ultrasonic waves in consideration of generation angle of grating lobes in the electronic sector scan system. For example, assuming that the sound speed in the living body is 1500 m/s, when the frequency of the transmission ultrasonic waves is 5 MHz, the wavelength thereof is about 0.3 mm, and the half of the wavelength of the ultrasonic waves is 0.15 mm. Accordingly, the arrangement pitch in the azimuth direction is 0.15 mm in the embodiment. On the other hand, the arrangement pitch in the elevation direction is 1.1 mm, which is larger than the wavelength of the transmission ultrasonic waves.

As shown in FIG. 1, in each element column, the elements 12 are connected to the switches SW1 to SW10 via the wirings 13. These switches SW1 to SW10 are provided for opening and closing the electric connections between respective adjacent two elements so as to form groups of elements. The number of switches is less than the number of elements in each element column (eleven) by one, and 1280 for 128 columns of elements. Further, the same control signal is supplied from the decoder 15 to the switches SW1 corresponding to the elements of the respective element columns, and that is similarly applicable to the switches SW2 to SW10. Each of the switches SW1 to SW10 is configured by an analog switch in combination of a P-channel MOSFET and an N-channel MOSFET, for example.

The serial/parallel converter circuit 14 receives a serial control signal CS and a clock signal CK from the ultrasonic examination apparatus main body 200, and converts the serial control signal into a parallel (e.g., 4-bit) control signal. The decoder 15 generates control signals to be supplied to the switches SW1 to SW10 based on the parallel control signal. Alternatively, the serial/parallel converter circuit 14 and the decoder 15 may be configured as an integrated circuit block.

The 128 sets of drive signals DS1 to DS5 to be supplied to the probe 100 via the respective coaxial cables are applied from the ultrasonic examination apparatus main body 200 to the plural element groups formed by the switches SW1 to SW10 in the respective 128 columns. Further, when ultrasonic waves are received, 128×5 reception signals are outputted from the plural element groups in the respective 128 columns via the respective coaxial cables to the ultrasonic examination apparatus main body 200.

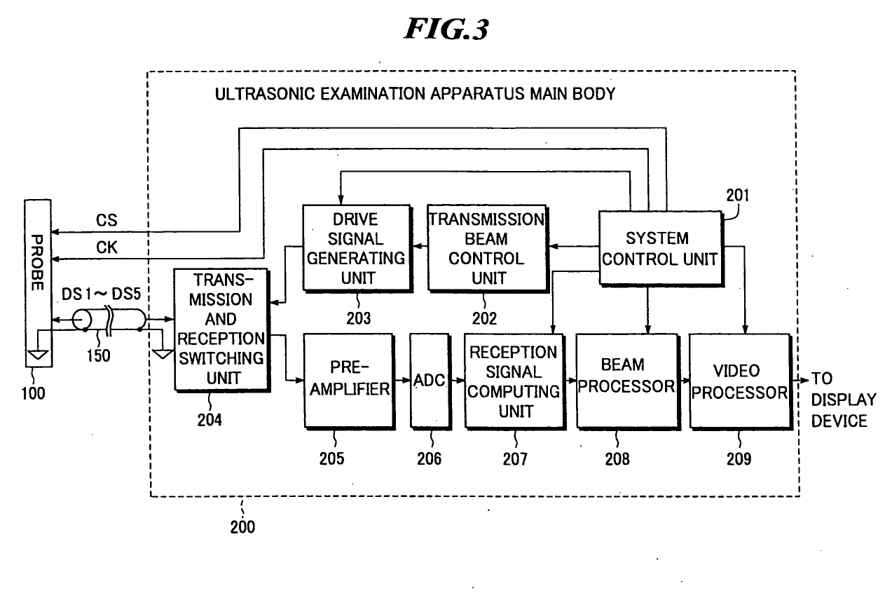

FIG. 3 is a block diagram for explanation of a configuration of the ultrasonic examination apparatus main body 200 shown in FIG. 1. As shown in FIG. 3, the ultrasonic examination apparatus main body 200 includes a system control unit 201 for controlling the respective parts of the ultrasonic examination apparatus, a transmission beam control unit 202, a drive signal generating unit 203, a transmission and reception switching unit 204, a preamplifier 205, an analog/digital converter (ADC) 206, a reception signal computing unit 207, a beam processor 208, and a video processor 209.

The system control unit 201 generates the control signal CS for controlling the switches SW1 to SW10 and supplies the control signal CS and the clock signal CK to the probe 100 in order to transmit and receive an ultrasonic beam in a desired direction and form a focal point at a desired depth.

The transmission beam control unit 202 sets the supply timing and delay times of the plural drive signals to be respectively supplied to the plural element groups under the control of the system control unit 201.

The drive signal generating unit 203 includes plural pulsers for generating the 128 sets of drive signals DS1 to DS5.

The transmission and reception switching unit 204 switches between the output of 128 sets of drive signals DS1 to DS5 to the probe 100 and the input of 128×5 reception signals from the probe 100. For passing the drive signals/reception signals between the ultrasonic examination apparatus main body 200 and the probe 100, 128×5=640 coaxial cables are used.

The preamplifier 205 preamplifies the reception signals outputted form the probe 100. Further, the A/D converter 206 converts the preamplified analog reception signals into digital reception signals (reception data). The preamplifier 205 and the A/D converter 206 are provided for 128×5 channels.

The reception signal computing unit 207 adjusts the levels of the acquired reception signals and performs phasing and addition processing thereon to generate reception data (sound ray data) corresponding to the transmission direction of the ultrasonic beam under the control of the system control unit 201.

The beam processor 208 performs predetermined signal processing such as envelope detection, STC (sensitivity time control), dynamic range adjustment, and filter processing on the reception data.

The video processor 209 converts the scan format with respect to the reception data on which the predetermined signal processing has been performed and further performs digital/analog conversion processing thereon to generate an analog video signal (image signal), and outputs the signal to a display device or the like.

Next, an operation of the ultrasonic examination apparatus according to the embodiment will be explained with reference to FIGS. 1-4B. As below, an operation with respect to one element column will be explained. Such an operation is also performed on elements of other element columns for scanning an ultrasonic beam in the azimuth direction. In this regard, a focal point of the ultrasonic beam can be formed at a desired depth in the azimuth direction as well by operating the elements of plural element columns while providing predetermined delay times in one transmission and reception of ultrasonic beam.

In the embodiment, a pseudo weighted arrangement, in which widths of elements differ depending on positions, is formed by grouping the plural elements 12 by using the switches SW1 to SW10. For the purpose, the system control unit 201 shown in FIG. 3 sets a switching pattern for controlling the operation of the switches SW1 to SW10 and a delay pattern of the drive signals to be supplied to the respective groups so as to be optimum according to the shape (transmission direction and focal length) of the ultrasonic beam.

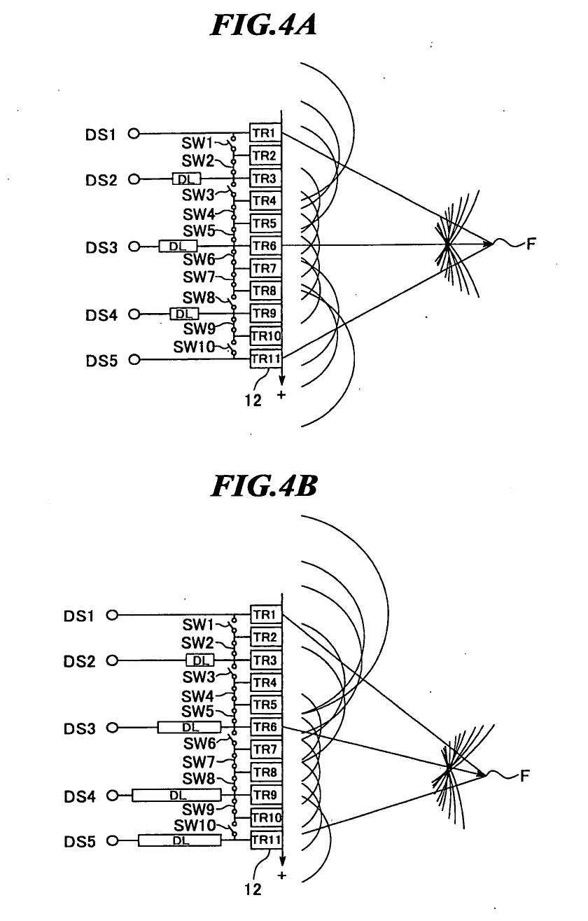

FIG. 4A shows connecting condition when an ultrasonic beam is transmitted and received in the frontward direction of the ultrasonic transducer array 10 (i.e., toward the central axis of the ultrasonic transducer array 10). In FIGS. 4A and 4B, the rightward direction relative to the transmission direction of the ultrasonic beam is the positive elevation direction.

In this case, the switches SW1, SW3, SW8, and SW10 are turned off and the switches SW2, SW4 to SW7, and SW9 are turned on. Thereby, element groups (TR1), (TR2, TR3), (TR4 to TRB), (TR9, TR10), and (TR11) are formed by the commonly connected elements 12. The drive signals provided with predetermined delay times DL are respectively supplied to these element groups via the drive signal supply lines DS1 to DS5. Here, the lengths of blocks “DL” shown on the respective drive signal supply lines DS1 to DS5 indicate the lengths of delay times to be supplied to the respective drive signals. According to the delay pattern, ultrasonic waves are sequentially transmitted from the element groups (TR1) and (TR11) at ends, and consequently, an ultrasonic beam is transmitted in the frontward direction of the ultrasonic transducer array 10 and a focal point “F” is formed at a predetermined depth.

FIG. 4B shows connecting condition when an ultrasonic beam is deflected and transmitted and received. Here, the “deflection” refers to transmission of ultrasonic beam in a direction away from the front direction. Further, a “deflection angle” refers to an angle formed by the transmission direction of the ultrasonic beam and the frontward direction.

As shown in FIG. 4B, when the ultrasonic beam is deflected by +10°, for example, the switches SW1, SW3, SW6, and SW10 are turned off and the switches SW2, SW4, SW5, and SW7 to SW9 are turned on. Thereby, element groups (TR1), (TR2, TR3), (TR4 to TR6), (TR7 to TR10), and (TR11) are formed by the commonly connected elements 12. The ultrasonic beam is transmitted in a direction at a deflection angle of 10°, for example, and a focal point F is formed at a predetermined depth by supplying the drive signals provided with predetermined delay times DL to these element groups via the drive signal supply lines DS1 to DS5 and sequentially driving them.

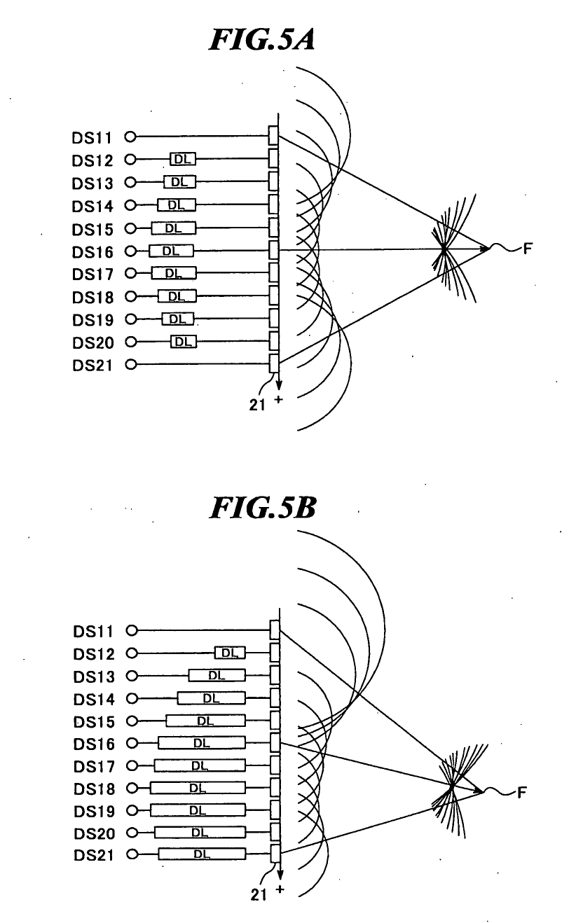

Here, advantages of changing the grouping of elements according to the transmission direction of ultrasonic beam in the embodiment will be explained with reference to FIGS. 5A-9.

FIGS. 5A and 5B are diagrams for explanation of a method of transmitting ultrasonic waves in a general phased array in which plural elements 21 are uniformly arranged (hereinafter, referred to as a uniform arrangement array). Drive signals DS11 to DS21 are supplied to these elements 21. In the phased array, as shown in FIG. 5A, when an ultrasonic beam is transmitted in the frontward direction, the delay pattern (delay times DL) is set such that the elements 21 are sequentially driven from ends to the center. Further, as shown in FIG. 5B, when an ultrasonic beam is deflected, the delay pattern is shifted such that the elements 21 farther from the deflection direction are driven earlier.

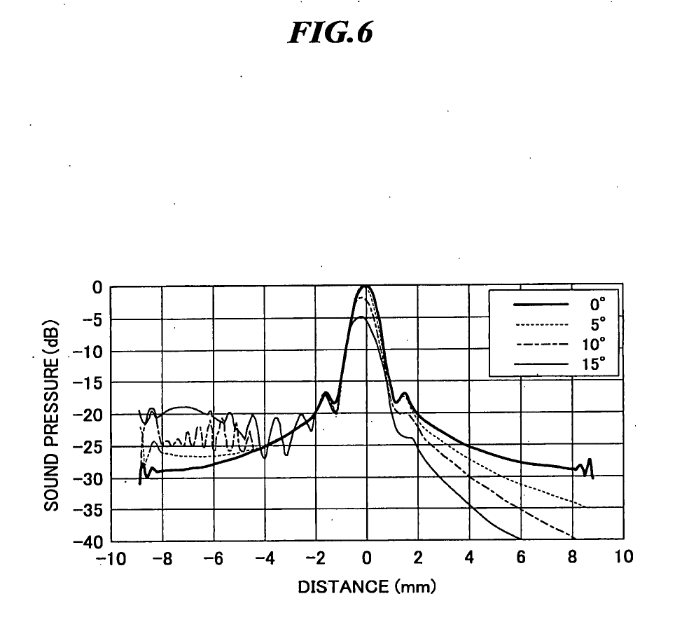

FIG. 6 shows a simulation result of profiles of ultrasonic beams transmitted from the phased array shown in FIGS. 5A and 5B. In FIG. 6, the horizontal axis indicates the distance from the central axis of the ultrasonic transducer array and the vertical axis indicates sound pressure (dB).

As shown in FIG. 6, in this case, generally good beam quality is obtained regardless of the deflection angle of the ultrasonic beam. However, in the phased array, coaxial cables are required for supplying drive signals in number corresponding to the number of elements, and there is a problem that the entire diameter of cables is larger.

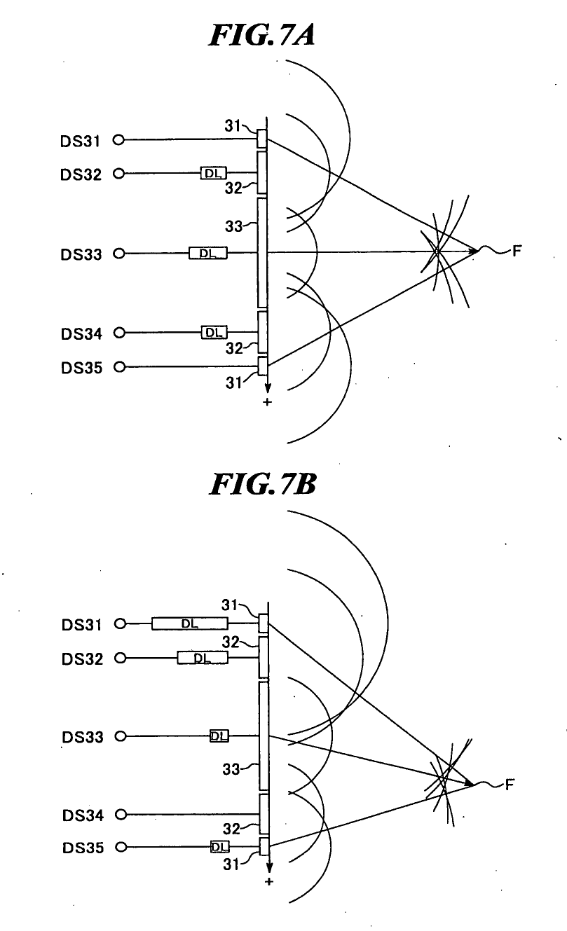

FIGS. 7A and 7B are diagrams for explanation of a method of transmitting ultrasonic waves in a phased array in which plural kinds of elements 31-33 are arranged in weighted arrangement (hereinafter, referred to as a weighted arrangement array). In the weighted arrangement array, in order to improve the quality of ultrasonic beam, the elements 31-33 are designed such that the widths of the elements are gradually narrower from the center to outer sides in the elevation direction. In the array, even when drive signals DS31 to DS35 are supplied to these elements 31-33, the problem of the entire diameter of cables is not caused because the number of element rows is small.

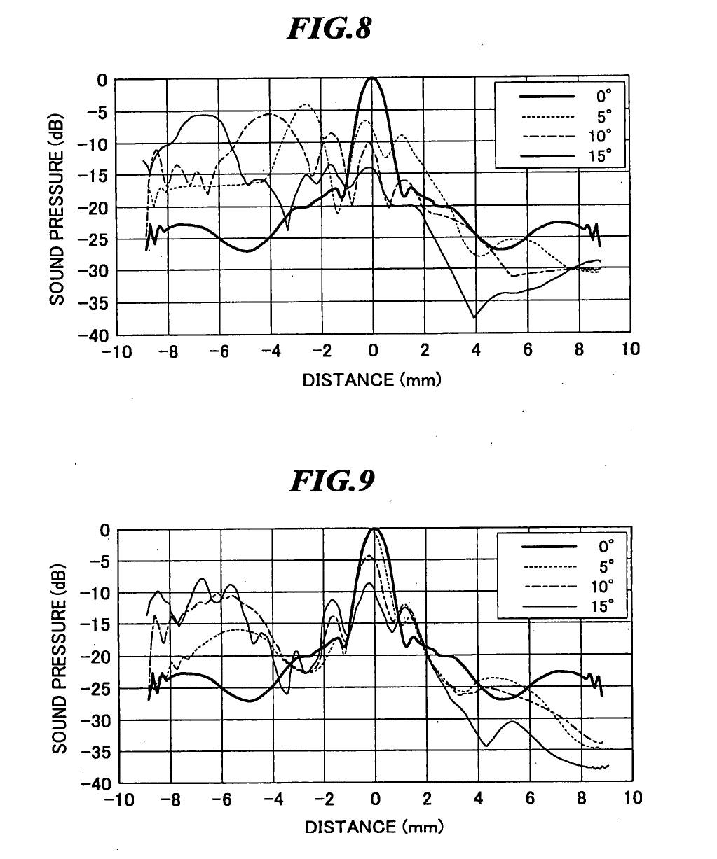

FIG. 8 shows a simulation result of profiles of ultrasonic beams transmitted from the weighted arrangement array shown in FIGS. 7A and 7B. As shown in FIG. 8, when an ultrasonic beam is transmitted in the frontward direction (deflection angle=0° as shown in FIG. 7A), the beam quality generally equal to that in the uniform arrangement array (FIG. 15) is obtained. However, if the ultrasonic beam is only slightly deflected (as shown in FIG. 7B), the ultrasonic beam quality is significantly deteriorated. For example, when the deflection angle is 5°, sidelobes at a higher sound pressure level than the main lobe appear near the distance −2 mm to −3 mm. This is because the weighted arrangement array shown in FIGS. 7A and 7B is designed such that the best beam quality can be obtained when the ultrasonic beam is transmitted in the frontward direction.

On the other hand, in the embodiment, when the ultrasonic beam is transmitted in the frontward direction, the element groups are formed such that the pseudo widths of the elements near the center are wider as is the case of the weighted arrangement array, on the other hand, when the ultrasonic beam is deflected, the element groups are formed such that the pseudo widths of the elements near the deflection direction are wider. Thereby, sidelobes when the ultrasonic beam is deflected can be reduced while the weighted arrangement is adopted by which the number of wirings can be reduced.

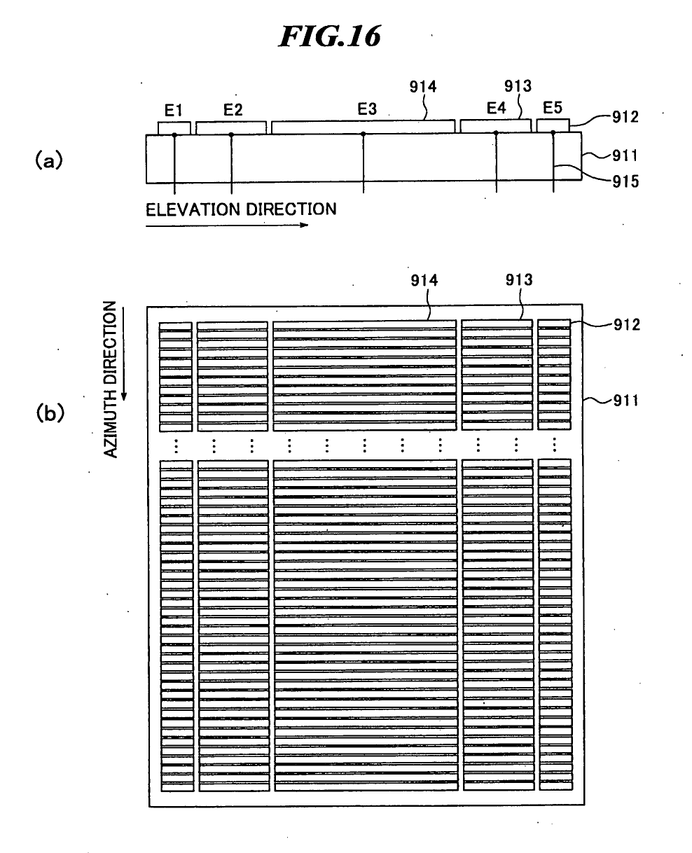

FIG. 9 shows a simulation result of profiles of ultrasonic beams transmitted from the ultrasonic examination apparatus (FIGS. 4A and 4B) according to the embodiment. As shown in FIG. 9, when an ultrasonic beam is transmitted in the frontward direction (deflection angle=0°), the beam quality generally equal to those in the uniform arrangement array (FIG. 15) and the weighted arrangement array (FIG. 16) can be obtained. Further, when the ultrasonic beam is deflected, although the level of sidelobes becomes slightly larger, a clear main lobe can be formed, and the beam quality can be significantly improved compared to that of the weighted arrangement array. When the deflection angle is 10° or more, relatively large sidelobes are observed in a location distant from the center (e.g., near −6 mm), however, the position is distant from the position of the main lobe, and the sidelobes do not have much effect on ultrasonic image information.

Here, since the amount of displacement of the piezoelectric material that forms the element 12 is determined by the voltage applied to the element 12, output sound pressure does not vary when the plural elements 12 having the same characteristic are connected in parallel as shown in FIGS. 4A and 4B. Further, since the output pressure of the element 12 is determined by the sound pressure received by the element 12, the voltages of the reception signals do not change when the plural elements 12 are commonly connected. Therefore, when the plural elements 12 are grouped, influence to the output sound pressure and reception sensitivity is small.

However, there is the following influence to the electric characteristic including the transmission and reception circuit.

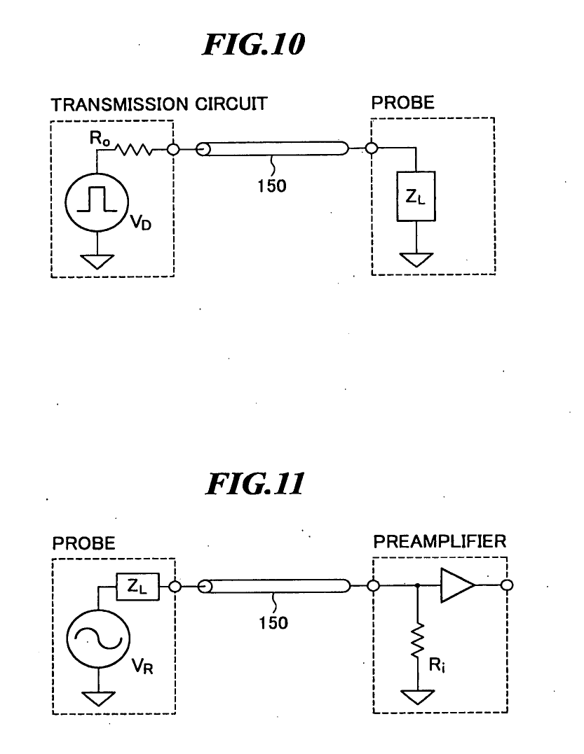

FIG. 10 shows an equivalent circuit of a transmission system circuit including the transmission circuit (pulser) in the drive signal generating unit 203 (FIG. 3) and the element in the probe. As shown in FIG. 10, the transmission circuit has a pulse signal source (drive voltage V_{D}) and an output impedance Rₒ. The transmission circuit and the element are connected by the coaxial cable 150. When ultrasonic waves are transmitted, a load impedance Z_{L} to the transmission circuit changes due to grouping of elements. Accordingly, when the output impedance Rₒ of the transmission circuit is not sufficiently small compared to the load impedance Z_{L}, the drive voltage V_{D} changes due to grouping of elements. Further, since the element is a capacitive load, the frequency characteristic of the transmission system circuit changes and the rising characteristic of the drive waveform also changes.

On the other hand, FIG. 11 shows an equivalent circuit of a reception system circuit including the element in the probe and the preamplifier 205 (FIG. 3). As shown in FIG. 11, at the time of reception, the element is equivalent to the signal source (reception voltage V_{R}) with the impedance Z_{L} of the element as an output impedance. Further, the preamplifier has an input impedance Rᵢ. Accordingly, a voltage value V_{R}·Rᵢ/(Z_{L}+Rᵢ) divided by the impedance Z_{L} of the element and the input impedance Rᵢ of the preamplifier is inputted to the preamplifier. Further, when the element impedance Z_{L} changes, the reflectance at the end of the coaxial cable 150 also changes.

On this account, when the elements are grouped and driven, the drive voltages drop or reception voltages drop depending on the number of commonly connected elements. Therefore, when the accuracy of ultrasonic image to be generated is made higher, it is desirable that these electric changes are corrected. Specifically, the system control unit 201 controls the units such that the voltage and/or waveform of the drive signals are corrected by the drive signal generating unit 203 (FIG. 3) and the gain and/or frequency characteristic of the reception system circuit are corrected by the reception signal computing unit 207 according to the switching pattern for grouping the elements.

As described above, according to the embodiment, the pseudo weighted arrangement array can be formed with the plural elements in uniform arrangement by grouping the plural elements in the respective channels of the multirow array. Therefore, the number of drive signal supply lines can be drastically reduced compared to that in the uniform arrangement array. Specifically, 128×11=1408 coaxial cables are required for a multirow array of 128 columns and 11 rows. In contrast, in the embodiment, since the eleven elements are divided into five groups, only 128×5=640 coaxial cables and two cables for supplying a control signal and a clock signal are required. Further, a ground line for logic circuit may be provided separately from the ground lines for analog circuits.

Further, since only (the number of elements −1) switches may be provided for the elements of the respective element columns, the total number of required switches in the ultrasonic transducer array is 1280. Therefore, the size of the probe is not so much upsized.

Furthermore, according to the embodiment, the pseudo weighted arrangement is changed according to the transmission direction of the ultrasonic beam, and thus, the good quality ultrasonic beam can be transmitted regardless of the direction. Therefore, good quality ultrasonic images can be generated based on the reception signals acquired by transmitting and receiving such ultrasonic beams.

In the above explanation, the case where the multirow array is disposed on a flat surface has been explained, however, a convex-type array or radial-type array may be formed by arranging plural elements on a curved surface formed by curving a flat surface or a side surface of a cylinder, for example.

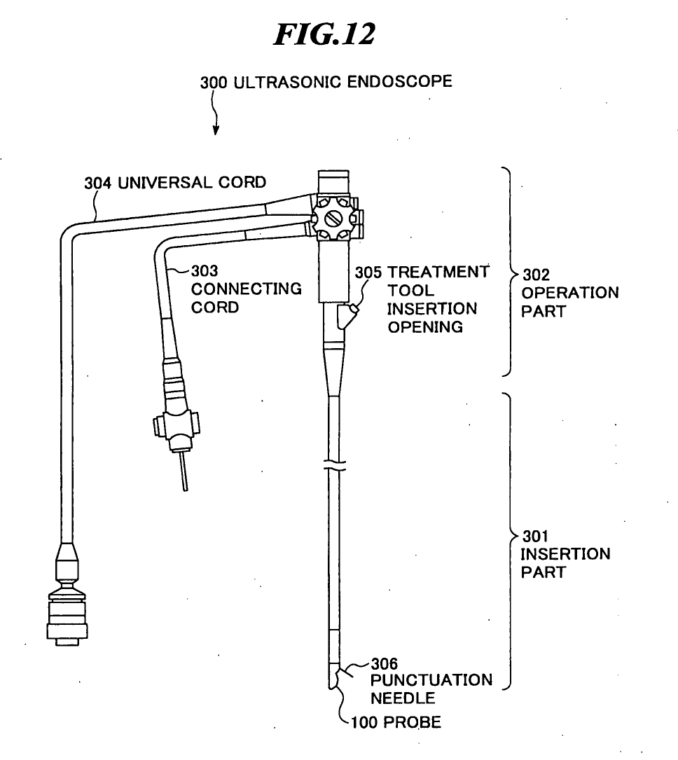

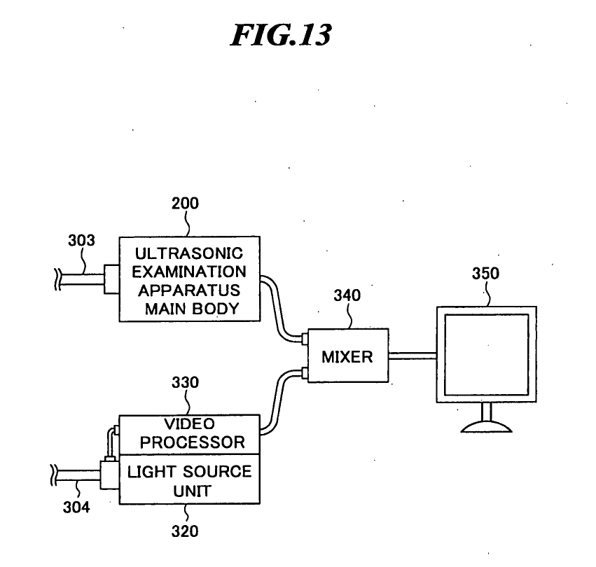

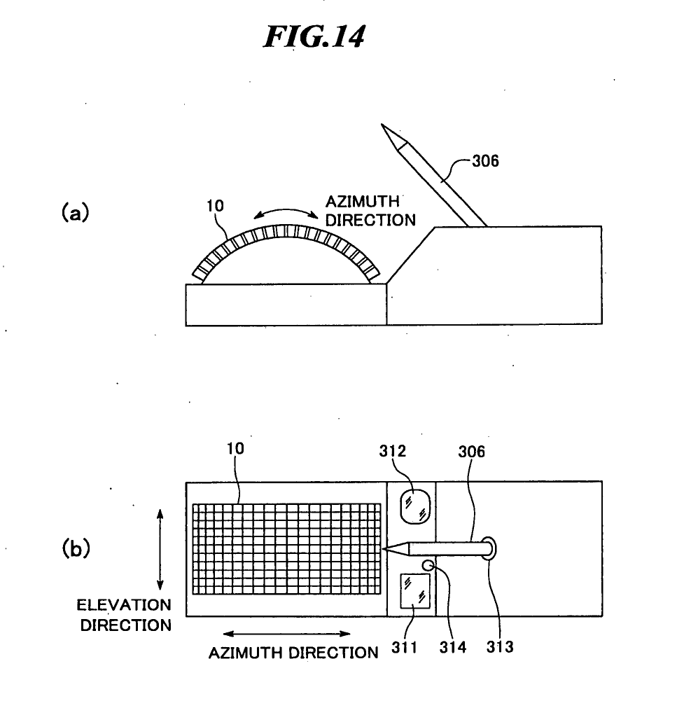

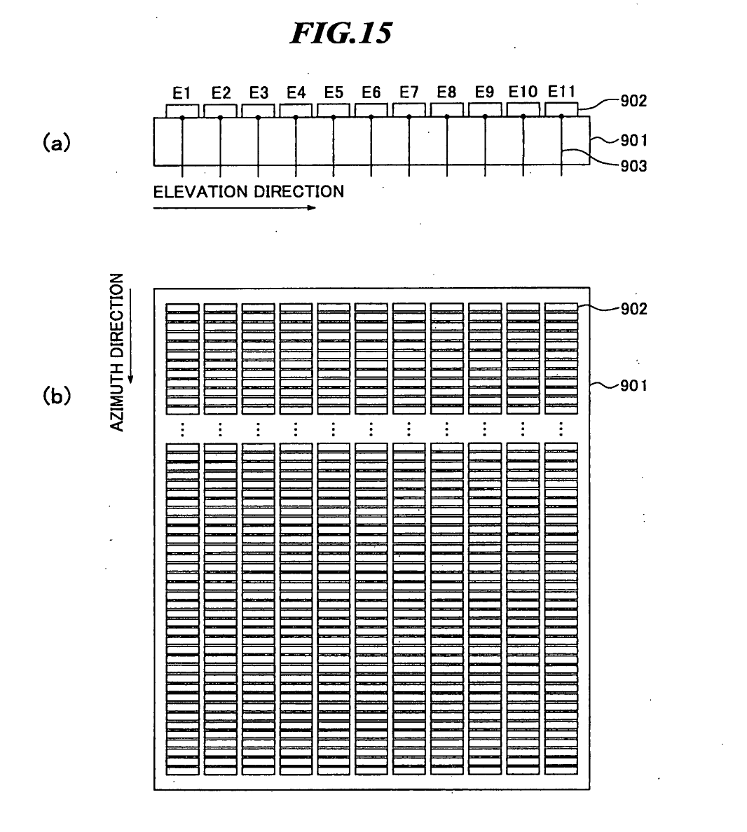

Next, a configuration of an ultrasonic endoscope examination apparatus to which the ultrasonic examination apparatus shown in FIG. 1 is applied will be explained with reference to FIGS. 12-14. FIG. 12 is a schematic diagram showing an appearance of the ultrasonic endoscope, FIG. 13 is a schematic diagram showing an apparatus connected to the ultrasonic endoscope shown in FIG. 12 for generating medical images, and FIG. 14 is an enlarged schematic diagram showing the leading end of an insertion part 301, that is, a probe 100 shown in FIG. 12.

As shown in FIG. 12, the ultrasonic endoscope 300 includes an insertion part 301, an operation part 302, a connecting cord 303, and a universal cord 304.

The insertion part 301 is an elongated tube formed of a material having flexibility for insertion into the body cavity of the object. The operation part 302 is provided at the base end of the insertion part 301, connected to the ultrasonic examination apparatus main body 200 shown in FIG. 13 via the connecting cord 303, and connected to a light source unit 320 shown in FIG. 13 via the universal cord 304.

The ultrasonic examination apparatus main body 200 shown in FIG. 13 supplies drive signals to the probe 100 shown in FIG. 12 to allow the probe 100 to transmit ultrasonic beams and generates ultrasonic image signals based on the reception signals outputted from the probe 100 when the probe 100 receives ultrasonic echoes.

The light source unit 320 generates light for illuminating the interior of the body cavity of the object. Further, a video processor 330 generates optical observation image signals representing the state within the object based on detection signals outputted from an image sensor provided at the leading end of the insertion part.

A mixer 340 generates image signals representing one of or both of an ultrasonic image and an optical observation image in one screen based on the ultrasonic image signals outputted from the ultrasonic examination apparatus 200 and the optical observation image signals outputted from the video processor 330 and outputs them to a display device 350. The display device 350 includes a display unit such as a CRT or LCD, and displays the ultrasonic image and/or the optical observation image based on the image signals outputted from the mixer 340.

FIG. 14(a) shows the leading end of the insertion part seen from the side, and FIG. 14(b) shows it seen from above. As shown in FIG. 14, at the leading end of the insertion part 301, that is, the probe 100 shown in FIG. 12, the ultrasonic transducer array 10, an observation window 331, an illumination window 312, a treatment tool passage opening 313, and a nozzle hole 314 are provided. Further, a punctuation needle 306 is provided in the treatment tool passage opening 313.

The ultrasonic transducer array 10 is a convex-type multirow array and includes eleven rows of elements arranged on a curved surface. Further, as shown in FIG. 14(b), it is desirable that the ultrasonic transducer array 10 is provided such that the elevation direction is perpendicular to the insertion direction of the treatment tool (e.g., the punctuation needle 306) provided in the treatment tool passage opening 313 as seen from above. Thereby, the position of the leading end of the treatment tool in the elevation direction can be detected. Though not shown, an acoustic matching layer is provided on the ultrasonic transmission face of the ultrasonic transducer array 10, and a backing layer is provided on the opposite face to the ultrasonic transmission face of the ultrasonic transducer array 10. In addition, an acoustic lens may be provided on the upper layer of the acoustic matching layer according to need.

An objective lens is fit in the observation window 311, and an input end of an image guide or a solid-state image sensor such as a CCD camera is provided in the imaging position of the objective lens. These configure an observation optical system, and the detection signals of the solid image sensor are outputted to the video processor 330 shown in FIG. 13. Further, an illumination lens for outputting illumination light to be supplied from the light source unit 320 shown in FIG. 13 via a light guide is fit in the illumination window 312. These configure an illumination optical system.

The treatment tool passage opening 313 is a hole for leading out a treatment tool inserted from a treatment tool insertion opening 305 (FIG. 12) provided in the operation part 302. Various treatments are performed within the living cavity of the object by projecting the treatment tool such as the punctuation needle 306 or forceps from the hole and operating it in the operation part 302. Furthermore, the nozzle hole 314 is provided for injecting a liquid (water or the like) for cleaning the observation window 311 and the illumination window 312.

1. An ultrasonic examination apparatus comprising:
a probe having a multirow array formed by arranging plural element rows in parallel with one another, each element row including one-dimensionally arranged ultrasonic transducers, and plural switches for opening and closing electric connections between respective adjacent two elements in each element column of said multirow array to form plural element groups;
control means for controlling the opening and closing of said plural switches according to a transmission/reception direction of an ultrasonic beam;
drive signal generating means for generating plural drive signals to be respectively supplied to said plural element groups; and
signal processing means for processing plural reception signals respectively outputted from said plural element groups to generate an image signal.

2. The ultrasonic examination apparatus according to claim 1, wherein said control means controls said drive signal generating means to change characteristics of the drive signals and/or controls said signal processing means to change characteristics of the reception signals according to the transmission/reception direction of the ultrasonic beam.

3. The ultrasonic examination apparatus according to claim 1, wherein said probe further has illuminating means and imaging means to be used for endoscope observation.

4. The ultrasonic examination apparatus according to claim 2, wherein said probe further has illuminating means and imaging means to be used for endoscope observation.
